(19) United States
(12) Patent Application Publication
(10) Pub. No.: US 2010/0087663 A1
(43) Pub. Date: Apr. 8, 2010

(54) POLYNARY METAL VANADIUM OXIDE PHOSPHATE

(75) Inventors: Hartmut Hibst, Schriesheim (DE); Robert Glaum, Rheinbach-Flerzheim (DE); Ernst Benser, Bonn (DE)

Correspondence Address: CONNOLLY BOVE LODGE & HUTZ LLP, P.O. BOX 2207, WILMINGTON, DE, 19899, US

(73) Assignee: Basf SE, Ludwigshafen (DE)

(21) Appl. No.: 12/531,537

(22) PCT Filed: Mar. 12, 2008
(86) PCT No.: PCT/EP08/52947

§ 371 Date: Sep. 16, 2009

(30) Foreign Application Priority Data
Mar. 16, 2007 (DE) 10 2007 012 722.9

(51) Int. Cl. C07D 307/33; B01J 27/198

(52) U.S. Cl. 549/256; 502/209

(57) ABSTRACT

A novel polynary metal oxide phosphate of the general formula I

MₐV₂O_{b}(PO₄)_{c}

is described, in which M is one or more metals selected from V, Cr, Fe, Co, Ni, Ru, Rh, Pd, Cu, Zn, Cd, Hg, Be, Mg, Ca, Sr and Ba, a is from 0.5 to 1.5, b is from 1.5 to 2.5, c is from 1.5 to 2.5, having a crystal structure whose powder X-ray diffractogram is characterized by defined reflections. Preferred representatives are CoV₂O₂(PO₄)₂, NiV₂O₂(PO₄)₂ or CuV₂O₂(PO₄)₂. The metal oxide phosphates are suitable as gas phase oxidation catalysts, for example for preparing maleic anhydride from a hydrocarbon having at least four carbon atoms.

The present invention relates to a polynaiy metal oxide phosphate which comprises vanadium and optionally at least one further metal, to a process for its preparation and to its use for heterogeneously catalyzed gas phase oxidations, preferably heterogeneously catalyzed gas phase oxidations of a hydrocarbon having at least four carbon atoms.

Heterogeneous catalysts based on vanadyl pyrophosphate (VO)₂P₂O₇ (so-called VPO catalysts) are used in the industrial oxidation of n-butane to maleic anhydride, and also in a series of further oxidation reactions of hydrocarbons.

The vanadyl pyrophosphate catalysts are generally prepared as follows: (1) synthesis of a vanadyl hydrogen phosphate hemihydrate precursor (VOHPO₄·½ H₂O) from a pentavalent vanadium compound (e.g. V₂O₅), a penta- or trivalent phosphorus compound (e.g. ortho- and/or pyrophosphoric acid, phosphoric esters or phosphorous acid) and a reducing alcohol (e.g. isobutanol), isolation of the precipitate, drying and optionally shaping (e.g. tableting) and (2) preforming the precursor to vanadyl pyrophosphate ((VO)₂P₂O₇) by calcining. Reference is made, for example, to EP-A 0 520 972 and WO 00/72963.

As a result of the use of an alcohol as a reducing agent, generally several % by weight of organic compounds remain included in the precursor and cannot be removed even by careful washing. In the further catalyst preparation, especially in the calcination, these exert an adverse effect on the catalytic properties of the catalyst. For instance, in the subsequent calcination, the risk exists of evaporation or of thermal decomposition of this included organic compound to form gaseous components which can lead to a pressure rise in the interior of the crystals and hence to destruction of the catalyst structure. This adverse effect is particularly marked in the case of calcination under oxidizing conditions, since the formation of the oxidized by-products, for example carbon monoxide or carbon dioxide, forms a significantly greater amount of gas. Furthermore, the oxidation of these organic compounds forms locally very large amounts of heat which can lead to thermal damage of the catalyst.

Moreover, the included organic compounds also have a significant influence on the adjustment of the local oxidation state of the vanadium. For instance, B. Kubias et al. in Chemie Ingenieur Technik 72 (3), 2000, pages 249-251 demonstrate the reducing effect of organic carbon in anaerobic calcination (under nonoxidizing conditions) of a vanadyl hydrogenphosphate hemihydrate precursor obtained from isobutanolic solution. In the example mentioned, anaerobic calcination afforded a mean oxidation state of the vanadium of 3.1, whereas aerobic calcination (under oxidizing conditions) afforded a mean oxidation state of the vanadium of about 4.

To improve the catalytic performance, it has been proposed to add small amounts of oxides of di-, tri- or tetravalent transition metals, known as promoters, to the vanadyl pyrophosphate (cf. G. J. Hutchings, J. Mater. Chem. 2004, 14, 3385-3395; K. V. Narayana et al., Z. Anorg. Allg. Chem. 2005, 631, 25-30). The mode of action of these promoters is to date substantially unexplained.

The literature to date does not include any information about the existence and the catalytic behavior of monophasic polynary vanadium(IV) phosphates which comprise a di-, tri- or tetravalent transition metal other than vanadium.

A mixed-valency vanadium(III,IV) diphosphate, V^{III}₂ (V^{IV}O)(P₂O₇)₂, has already been known for some time and also characterized by crystallographic means; cf. J. W. Johnson et al., Inorg. Chem. 1988, 27, 1646-1648. B. G. Golovkin, V. L. Volkov, Russ. J. Inorg. Chem. 1987, 32, 739-741 discloses a further compound which has likewise been described as the diphosphate V₃O₄(P₂O₇); however, there is a complete lack of information on its characterization.

It was an object of the present invention to provide novel polynary vanadium oxide phosphates.

It was a further object of the present invention to provide novel polynary vanadium oxide phosphates with catalytic properties for heterogeneously catalyzed gas phase oxidations.

It was a further object of the present invention to provide novel polynary vanadium oxide phosphates with whose aid the catalytic properties of known heterogeneous catalysts based on vanadyl pyrophosphate can be modified.

Further objects of the invention related to the provision of processes for preparing the novel polynary vanadium oxide phosphates and processes for heterogeneously catalyzed gas phase oxidation.

Accordingly, a polynaiy metal oxide phosphate of the general formula I

MₐV₂O_{b}(PO₄)_{c}

has been found, in which

M is one or more metals selected from V, Cr, Fe, Co, Ni, Ru, Rh, Pd, Cu, Zn, Cd, Hg, Be, Mg, Ca, Sr and Ba,

a is from 0.5 to 1.5,

b is from 1.5 to 2.5,

c is from 1.5 to 2.5,

having one of the two following crystal structures A or B where

the powder X-ray diffractogram of crystal structure A is characterized by reflections at the interplanar spacings d [Å]=6.28±0.06, 4.75±0.04, 331±0.04, 3.14±0.04, 2.60±0.04 and

the powder X-ray diffractogram of crystal structure B is characterized by reflections at the interplanar spacings d [Å]=5.81±0.06, 4.77±0.04, 4.55±0.04, 3.84±0.04, 3.28±0,04, 3.17±0.04, 2.77±0,04, 2.70±0.04.

In this application, the X-ray reflections are reported in the form of the interplanar spacings d [Å] which are independent of the wavelength of the X-radiation used. The wavelength λ of the X-radiation used for diffraction and the diffraction angle θ (in this document, the reflection position used is the peak location of a reflection in the 2θ plot) are linked to one another via the Bragg equation as follows:

2 sin θ=λ/d

where d is the interplanar spacing of the atomic three-dimensional arrangement corresponding to the particular reflection.

The powder X-ray diffractogram of the inventive metal oxide phosphate of the formula I is characterized by reflections according to one of the two lists A and B which follow.

The reflections of list A generally have the approximate relative intensities (Iᵣₑₗ [%]) specified in Table 1. Further, generally less intensive reflections of the powder X-ray diffractogram have not been included in Table 1.

TABLE 1

| d [Å] | Rel. intensity [%] |
|---|---|
| 6.28 ± 0.06 | 25 ± 15 |
| 4.75 ± 0.04 | 30 ± 20 |
| 3.31 ± 0.04 | 100 |
| 3.14 ± 0.04 | 45 ± 25 |
| 2.60 ± 0.04 | 25 ± 15 |

The reflections of list B generally have the approximate relative intensities (Iᵣₑₗ [%]) specified in Table 2. Further, generally less intensive reflections of the powder X-ray diffractogram have not been included in Table 2.

TABLE 2

| d [Å] | Rel. intensity [%] |
|---|---|
| 5.81 ± 0.06 | 25 ± 15 |
| 4.77 ± 0.04 | 25 ± 15 |
| 4.55 ± 0.04 | 15 ± 10 |
| 3.84 ± 0.04 | 15 ± 10 |
| 3.28 ± 0.04 | 100 |
| 3.17 ± 0.04 | 35 ± 20 |
| 2.77 ± 0.04 | 15 ± 10 |
| 2.70 ± 0.04 | 25 ± 15 |

Depending on the crystallinity and the texture of the resulting crystals of the inventive metal oxide phosphate, however, there may be enhancement or attenuation of the intensity of the reflections in the powder X-ray diffractogram, The attenuation may be to such an extent that individual reflections in the powder X-ray diffractogram are no longer detectable.

It is self-evident to the person skilled in the art that mixtures of the inventive metal oxide phosphates with other crystalline compounds have additional reflections. Such mixtures of the metal oxide phosphate with other crystalline compounds can be prepared in a controlled manner by mixing the inventive metal oxide phosphate or can be formed in the preparation of the inventive metal oxide phosphates by incomplete conversion of the starting materials or formation of extraneous phases with different crystal structure.

In the formula I, a is preferably from 0.8 to 1.2, especially about 1.

In formula I, b is preferably from 1.8 to 2.2, especially about 2.

In formula I, c is preferably from 1.8 to 12, especially about 2.

In formula I, M is a metal selected from V, Cr, Fe, Co, Ni, Ru, Rh, Pd, Cu, Zn, Cd, Hg, Be, Mg, Ca, Sr and Ba, or combinations of two or more of these metals. M is preferably a metal selected from Co, Ni and Cu.

Particularly preferred inventive metal oxide phosphates have one of the following formulae:

CoV₂O₂(PO₄)₂,

NiV₂O₂(PO₄)₂ or

CuV₂O₂(PO₄)₂.

The inventive metal oxide phosphates are obtainable in various ways.

Firstly, the inventive metal oxide phosphates can be obtained by a solid-state reaction in a closed system. For this purpose, at least two reactants selected from oxygen compounds of vanadium, phosphorus compounds of vanadium and mixed oxygen-phosphorus compounds of vanadium, elemental vanadium, oxygen compounds of the metal M, phosphorus compounds of the metal M and mixed oxygen-phosphorus compounds of the metal M and elemental metal M are reacted.

In this case, the reactants are generally selected such that (i) they provide the desired stoichiometry of the elements in the formula I and (ii) the sum of the products of valency multiplied by frequency of the elements other than oxygen in the reactants corresponds to the sum of the products of valency multiplied by frequency of the elements other than oxygen in the formula I. The starting compounds may be selected such that all elements other than oxygen therein already possess the valency that they possess in the formula I. Alternatively, the starting compounds can be selected such that some or all elements other than oxygen therein possess a valency different from that which they possess in formula I. As a result of redox reactions, for example a synproportionation, during the solid-state reaction, the elements other than oxygen receive the valency which they possess in the formula I. For example, it is possible to use a combination of equivalent amounts of vanadium(III) and vanadium(V) compounds from which tetravalent vanadium forms in the solid-state reaction.

The solid-state reaction proceeds, for example, according to one of the following equations (1) or (2):

(VO)₂P₂O₇+MO→MV₂O₂(PO₄)₂ (e.g. M=Co, Ni, Cu) (1)

M₂P₄O₁₂+4 VO₂→2 MV₂O₂(PO₄)₂ (e.g. M=Co, Ni, Cu) (2)

The starting compounds required, in the form of oxides, phosphates, oxide phosphates, phosphides or the like, are either commercially available or known from the literature or can be synthesized easily by the person skilled in the art in analogy to known preparation methods.

The starting materials are mixed intimately, for example by fine trituration. The solid-state reaction is effected typically at a temperature of at least 500° C., for example from 650 to 1100° C., especially about 800° C. Typical reaction times are, for example, from 24 hours to 10 days. Suitable reaction vessels consist, for example, of quartz glass or corundum.

In order to obtain products with a high crystallinity or single crystals, it is appropriately possible in the solid-state reaction to use a suitable mineralizer, such as iodine or PtCl₂.

Alternatively, inventive metal oxide phosphates can be prepared by
a) preparing a dry mixture of a vanadium source, of a source of the metal M and of a phosphate source,
b) optionally providing reduction equivalents in order to convert the vanadium and/or the metal M to the valency state possessed by the vanadium and the metal Min the formula and
c) calcining the dry mixture at at least 500° C.

To this end, a mixture of suitable sources of the elemental constituents of the metal oxide phosphates is used to obtain a very intimate, preferably finely divided, dry mixture of the desired constituent stoichiometry.

The starting compounds can be mixed intimately in dry or in wet form.

When it is effected in dry form, the starting compounds are appropriately used as finely divided powders and, after the mixing and optional compaction, subjected to calcination (thermal treatment).

However, preference is given to effecting the intimate mixing in wet form, i.e. in dissolved or suspended form. The starting compounds are typically mixed with one another in the form of an aqueous solution (optionally with use of complexing agents) and/or suspension. Subsequently, the aqueous solution or suspension is dried and, after the drying, calcined.

The drying can be effected by evaporation under reduced pressure, by freeze-drying or by conventional evaporation. However, preference is given to effecting the drying process by spray-drying. The exit temperatures are generally from 70 to 150° C.; the spray-drying can be performed in cocurrent or in countercurrent.

Suitable vanadium sources are, for example, vanadyl sulfate hydrate, vanadyl acetylacetonate, vanadates such as ammonium metavanadate, vanadium oxides, for example divanadium pentoxide (V₂O₅), vanadium dioxide (VO₂) or divanadium trioxide (V₂O₃), vanadium halides, for example vanadium tetrachloride (VCl₄) and vanadyl halides, for example VOCl₃. Divanadium pentoxide and ammonium vanadate are preferred vanadium sources.

Useful sources for the metal M include all compounds of the elements which are capable of forming oxides and/or hydroxides when heated (optionally in the presence of molecular oxygen, for example under air). Of course, the starting compounds of this type which are used may also partly or exclusively already be oxides and/or hydroxides of the elemental constituents. The source of the metal M is preferably selected from nitrates, carboxylates, carbonates, hydrogencarbonates, basic carbonates, oxides, hydroxides and oxide hydroxides of the metal M.

Suitable phosphate sources are compounds comprising phosphate groups or compounds from which phosphate groups form by redox reactions and/or in the course of heating (optionally in the presence of molecular oxygen, for example under air). These include phosphoric acids, especially orthophosphoric acid, pyro- or metaphosphoric acids, phosphorous acid, hypophosphorous acid, phosphates or hydrogenphosphates such as diammonium hydrogenphosphate, and elemental phosphorus, for example white phosphorus. The phosphate source is preferably formed at least partly by phosphorous acid or hypophosphorous acid, optionally in combination with orthophosphoric acid.

In one embodiment, the dry mixture is prepared by mixing vanadyl hydrogenphosphate hemihydrate with a source of the metal M, which is suitably selected from nitrates, carboxylates, carbonates, hydrogencarbonates, basic carbonates, oxides, hydroxides and oxide hydroxides of the metal M.

When the vanadium sources or sources for the metal M used are compounds in which the vanadium or the metal M has a higher valency than it possesses in the formula I (i.e. than the formal valency of V and any M which is required to obtain electrical neutrality with the O²⁻ and PO₄³⁻ anions present in formula I), reduction equivalents should preferably be provided in order to convert the vanadium and/or the metal M to the valency state that the vanadium and the metal M possess in the formula I.

The reduction equivalents are provided by a reducing agent which is capable of reducing the higher-valency form of the vanadium or of the metal M. The reduction can be effected in the course of preparation of the dry mixture or in the course of calcination at the latest, Preference is given to preparing the intimate dry mixture under inert gas atmosphere (e.g. N₂) in order to ensure better control over the oxidation states.

Preferred reducing agents for this purpose are selected from hypophosphorous acid, phosphorous acid, hydrazine (as the free base or hydrate or in the form of its salts such as hydrazine dihydrochloride, hydrazine sulfate), hydroxylamine (as the free base or in the form of its salts such as hydroxylamine hydrochloride), nitrosylamine, elemental vanadium, elemental phosphorus, borane (including in the form of complex borohydrides such as sodium borohydride) or oxalic acid. Phosphorous acid and/or hypophosphorous acid are preferred reducing agents.

It is self-evident that particular reducing agents such as hypophosphorous acid or phosphorous acid can simultaneously serve as the phosphate source, or elemental vanadium simultaneously serves as the vanadium source.

The dry mixture is treated thermally at temperatures of at least 500° C., preferably from 700 to 1000° C., especially about 800° C. The thermal treatment can be effected under an oxidizing, reducing or inert atmosphere. Useful oxidizing atmosphere includes, for example, air, air enriched with molecular oxygen or air depleted of oxygen. However, preference is given to performing the thermal treatment under inert atmosphere, i.e., for example, under molecular nitrogen and/or noble gas. The thermal treatment is typically effected at standard pressure (1 atm). Of course, the thermal treatment can also be effected under reduced pressure or under elevated pressure.

When the thermal treatment is effected under gaseous atmosphere, the latter may either be stationary or flow. It preferably flows. Overall, the thermal treatment may take up to 24 h or more.

The invention further relates to a gas phase oxidation catalyst which comprises at least one inventive polynary metal oxide phosphate. The metal oxide phosphates may be used as such, for example as powders, or in the form of shaped bodies as heterogeneous catalysts.

Preference is given to effecting the shaping by tableting. For tableting, a tableting assistant is generally added to the powder and mixed intimately.

Tableting assistants are generally catalytically inert and improve the tableting properties of the powder, for example by increasing the lubrication and free flow. Suitable and preferred tableting assistants include graphite or boron nitride. The tableting assistants added generally remain in the activated catalyst.

The powder can also be tableted and subsequently comminuted to spall.

The shaping to shaped bodies can, for example, also be effected by applying at least one inventive metal oxide phosphate or mixtures which comprise at least one inventive metal oxide phosphate to a support body.

The support bodies are preferably chemically inert. hi other words, they essentially do not intervene in the course of the catalytic gas phase oxidation which is catalyzed by the inventive metal oxide phosphates.

Useful materials for the support bodies include especially aluminum oxide, silicon dioxide, silicates such as clay, kaolin, steatite, pumice, aluminum silicate and magnesium silicate, silicon carbide, zirconium dioxide and thorium dioxide.

The surface of the support body may either be smooth or rough. Advantageously, the surface of the support body is rough, since an increased surface roughness generally causes an increased adhesion strength of the applied active composition coating.

Moreover, the support material may be porous or nonporous. The support material is appropriately nonporous, i.e. the total volume of the pores is preferably less than 1% by volume, based on the volume of the support body.

The thickness of the catalytically active layer is typically from 10 to 1000 μm, for example from 50 to 700 μm, from 100 to 600 or from 150 to 400 μm.

In principle, support bodies with any geometric structure are useful. Their longest dimension is generally from 1 to 10 mm. However, preference is given to employing spheres or cylinders, especially hollow cylinders, as support bodies.

In the simplest manner, the coated catalysts can be prepared by preforming metal oxide phosphate compositions of the general formula (I), converting them to finely divided form and finally applying them to the surface of the support body with the aid of a liquid binder. To this end, the surface of the support body is, in the simplest manner, moistened with the liquid binder, and a layer of the active composition is adhered on the moistened surface by contacting it with the finely divided metal oxide phosphate composition. Finally, the coated support body is dried. Of course, the operation can be repeated to achieve a greater layer thickness.

The inventive metal oxide phosphates may also be used in order to modify the catalytic properties, especially conversion and/or selectivity, of known catalysts, especially based on vanadyl pyrophosphate. To this end, the inventive metal oxide phosphates may be used, for example, as a promoter phase in a catalyst based on vanadyl pyrophosphate. Appropriately, the catalyst then comprises a first phase and a second phase in the form of three-dimensional regions which are delimited from their local environment by a different chemical composition. In this case, the first phase comprises a catalytically active material based on vanadyl pyrophosphate and the second phase at least one inventive polynary metal oxide phosphate. In this case, (i) finely divided particles of the second phase may be dispersed in the first phase, or (ii) the first phase and the second phase may be distributed relative to one another as in a mixture of finely divided first phase and finely divided second phase.

These biphasic catalysts can be prepared, for example, by preparing a vanadyl hydrogenphosphate hemihydrate precursor (VOHPO₄·½ H₂O), admixing it with preformed particles of the second phase of inventive metal oxide phosphate, shaping the resulting material and calcining it. The vanadyl hydrogenphosphate hemihydrate precursor can be obtained in a manner known per se from a compound of pentavalent vanadium (e.g. V₂O₅), a compound comprising penta- or trivalent phosphorus (e.g. ortho- and/or pyrophosphoric acid, phosphoric ester or phosphorous acid) and a reducing alcohol (e.g. isobutanol), and isolating the precipitate. Reference is made, for example, to EP-A 0 520 972 and WO 00/72963.

The inventive catalysts whose catalytically active composition comprises at least one above-defined metal oxide phosphate may also be combined with catalysts based on vanadyl pyrophosphate in the form of a structured packing. For instance, a gas stream which comprises a hydrocarbon to be oxidized and molecular oxygen can be passed through a bed of a first gas phase oxidation catalyst placed upstream in flow direction of the gas stream and then through one or more downstream beds of a second or further gas phase oxidation catalysts, in which case the first or second or one of the further beds comprises an inventive catalyst.

The invention further relates to a process for partial gas phase oxidation or ammoxidation, in which a gas stream which comprises a hydrocarbon and molecular oxygen is contacted with an inventive catalyst. In the case of ammoxidation, the gas stream additionally comprises ammonia. In the context of the present invention, ammoxidation is understood to mean a heterogeneously catalyzed process in which methyl-substituted alkenes, arenes and hetarenes are converted to nitriles by reaction with ammonia and oxygen in the presence of transition metal catalysts.

In preferred embodiments, the process for partial gas phase oxidation serves to prepare maleic anhydride, in which case the hydrocarbon used comprises at least four carbon atoms.

In the process according to the invention for partial gas phase oxidation or ammoxidation, generally tube bundle reactors are used. Alternatively, it is also possible to use fluidized bed reactors.

Suitable hydrocarbons are generally aliphatic and aromatic, saturated and unsaturated hydrocarbons having at least four carbon atoms, for example 1,3-butadiene, 1-butene, cis-2-butene, trans-2-butene, n-butane, C₄ mixtures, 1,3-pentadiene, 1,4-pentadiene, 1-pentene, cis-2-pentene, trans-2-pentene, n-pentane, cyclopentadiene, dicyclopentadiene, cyclopentene, cyclopentane, C₅ mixtures, hexenes, hexanes, cyclohexane and benzene. Preference is given to using 1,3-butadiene, 1-butene, cis-2-butene, trans-2-butene, n-butane, benzene or mixtures thereof.

Particular preference is given to the use of n-butane and n-butane-containing gases and liquids. The n-butane used may stem, for example, from natural gas, from steam crackers or FCC crackers.

The hydrocarbon is generally added under quantitative control, i.e. with constant specification of a defined amount per unit time. The hydrocarbon can be metered in in liquid or gaseous form. Preference is given to metered addition in liquid form with subsequent evaporation before entry into the reactor.

The oxidizing agents used are oxygen-comprising gases, for example air, synthetic air, a gas enriched with oxygen or else so-called “pure” oxygen, i.e. oxygen stemming, for example, from air fractionation. The oxygen-comprising gas is preferably also added under quantitative control.

The gas to be passed through the reactor generally comprises a hydrocarbon concentration of from 0.5 to 15% by volume and an oxygen concentration of from 8 to 25% by volume. The proportion lacking from 100% by volume is composed of further gases, for example nitrogen, noble gases, carbon monoxide, carbon dioxide, steam, oxygenated hydrocarbons (e.g. methanol, formaldehyde, formic acid, ethanol, acetaldehyde, acetic acid, propanol, propionaldehyde, propionic acid, acrolein, crotonaldehyde) and mixtures thereof. In the case of selective oxidation of n-butane, the n-butane content in the total amount of hydrocarbon is preferably more than 90% and more preferably more than 95%.

To ensure a long catalyst lifetime and further increase in conversion, selectivity, yield, catalyst hourly space velocity and space-time yield, a volatile phosphorus compound is preferably added to the gas in the process according to the invention.

At the start, i.e. at the reactor inlet, its concentration is at least 0.2 ppm by volume, i.e. 0.2×10⁻⁶ parts by volume of the volatile phosphorus compounds based on the total volume of the gas at the reactor inlet. Preference is given to a content of from 0.2 to 20 ppm by volume, particular preference to a content of from 0.5 to 10 ppm by volume.

Volatile phosphorus compounds are understood to mean all of those phosphorus-comprising compounds which are present in gaseous form under the use conditions in the desired concentration. Examples of suitable volatile phosphorus compounds include phosphines and phosphoric esters. Particular preference is given to the C₁- to C₄-alkyl phosphates, very particular preference to trimethyl phosphate, triethyl phosphate and tripropyl phosphate, especially triethyl phosphate.

The process according to the invention is performed generally at a temperature of from 300 to 500° C. The temperature mentioned is understood to mean the temperature of the catalyst bed present in the reactor which would be present when the process is executed in the absence of a chemical reaction.

When this temperature is not exactly the same at all points, the term means the numerical average of the temperatures along the reaction zone. In particular, this means that the true temperature present over the catalyst, owing to the exothermicity of the oxidation reaction, may also be outside the range mentioned. Preference is given to performing the process according to the invention at a temperature of from 380 to 460° C., more preferably from 380 to 430° C.

The process according to the invention can be executed at a pressure below standard pressure (for example up to 0.05 MPa abs) or else above standard pressure (for example up to 10 MPa abs). This is understood to mean the pressure present in the reactor unit. Preference is given to a pressure of from 0.1 to 1.0 MPa abs, particular preference to a pressure of from 0.1 to 0.5 MPa abs.

The process according to the invention can be performed in two preferred process variants, the variant with “straight pass” and the variant with “recycling”. In “straight pass”, maleic anhydride and any oxygenated hydrocarbon by-products are removed from the reactor effluent and the remaining gas mixture is discharged and optionally utilized thermally. In the case of “recycling”, maleic anhydride and any oxygenated hydrocarbon by-products are likewise removed from the reactor effluent, the remaining gas mixture which comprises unconverted hydrocarbon is recycled fully or partly to the reactor. A further variant of “recycling” is the removal of the unconverted hydrocarbon and the recycling thereof to the reactor.

In a particularly preferred embodiment for preparation of maleic anhydride, n-butane is used as the starting hydrocarbon and the heterogeneously catalyzed gas phase oxidation is performed in “straight pass” over the inventive catalyst.

The present invention is illustrated in detail by the appended figures and the examples which follow.

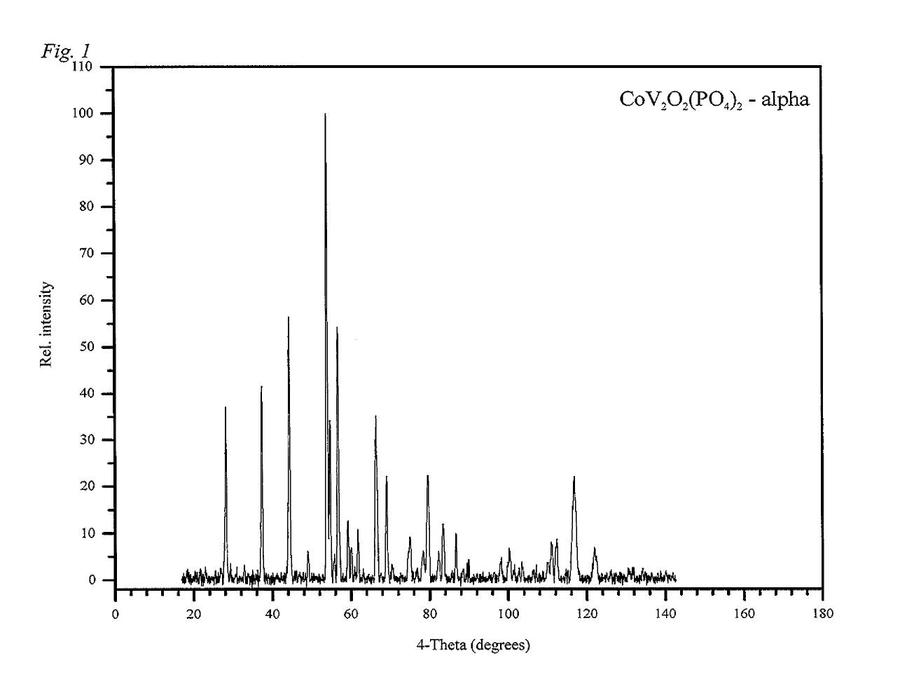

FIG. 1 shows a Guinier image of α-CoV₂O₂(PO₄)₂ which has been obtained by solid-state reaction;

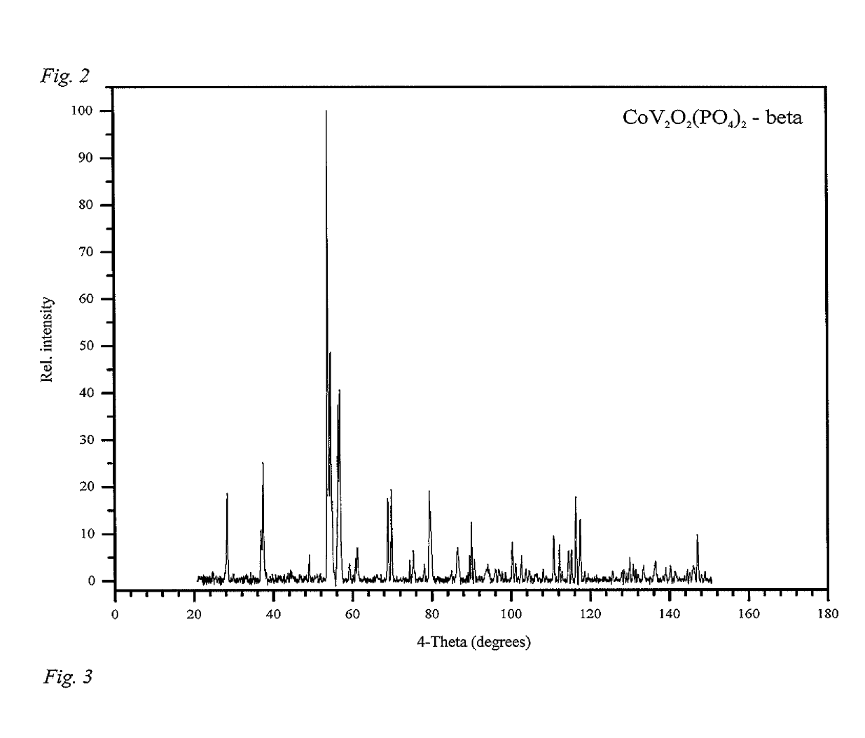

FIG. 2 shows a Guinier image of β-CoV₂O₂(PO₄)₂ which has been obtained by solid-state reaction;

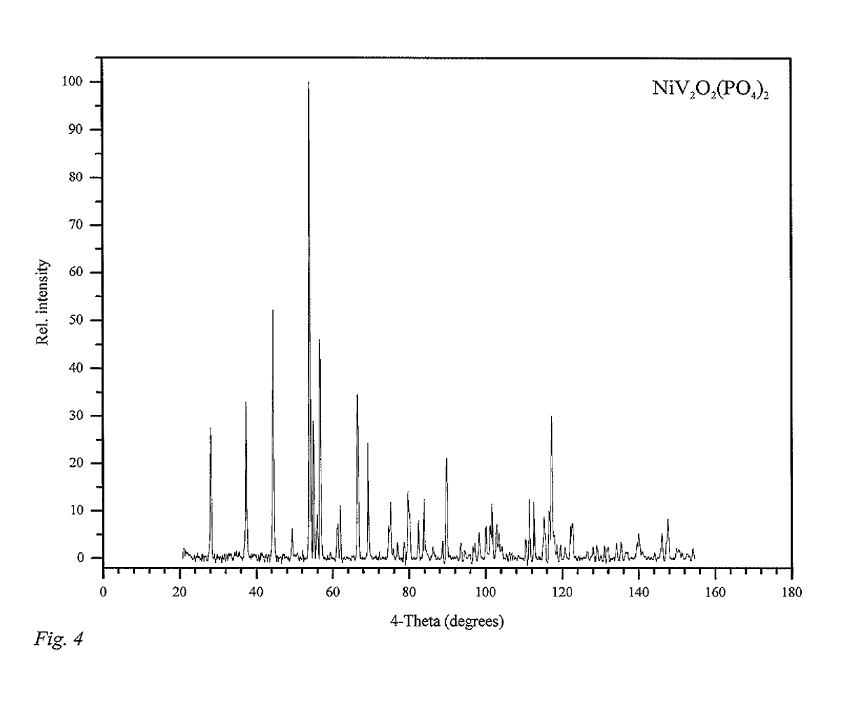

FIG. 3 shows a Guinier image of NiV₂O₂(PO₄)₂ which has been obtained by solid-state reaction;

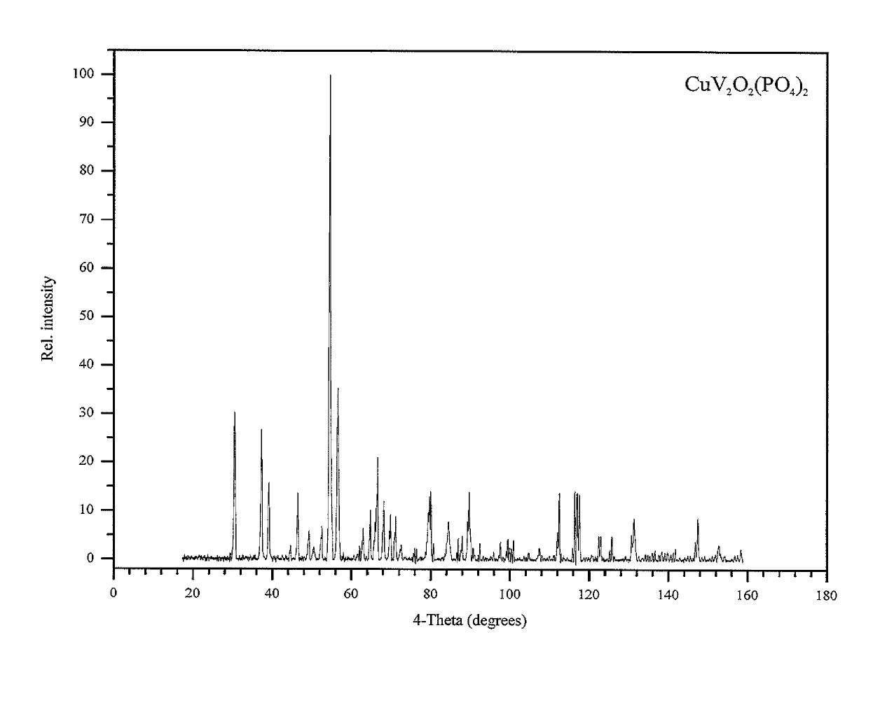

FIG. 4 shows a Guinier image of CuV₂O₂(PO₄)₂ which has been obtained by solid-state reaction.

For the X-ray diffraction analyses by Guinier technology, an FR-552 camera (from Nonius, Delft) was utilized using image plate film (Y. Amemiya, J. Miyahara, NATURE 1988, 336, 89-90) (CuKα₁ radiation, λ=1.54051 Å, α-quartz monochromator, α-SiO₂ as the internal standard). See K. Maaβ, R. Glaum, R. Gruehn, Z. anorg. Allg. Chem. 2002, 628, 1663-1672.

All other X-ray diffraction analyses are based on X-ray diffractograms obtained using Cu-Kα radiation (λ=1.54178 Å) as X-radiation (Theta-Theta D-5000 Siemens diffractometer, tube voltage: 40 kV, tube current: 40 mA, aperture V20 (variable), collimator V20 (variable), secondary monochromator aperture (0.1 mm), detector aperture (0.6 mm), measurement interval (2θ): 0.02[°], measurement time per step: 2.4 s, detector: scintillation counting tube).

### EXAMPLE 1

### Preparation of α-CoV₂O₂(PO₄)₂ by Solid-State Reaction

First, (VO)₂P₂O₇ was prepared by heating VO(HPO₄)·½ H₂O in an argon stream at 1073 K (J. W. Johnson, D. C. Johnston, A. J. Jacobson, J. F. Brody, J. Amer. Chem. Soc. 1984, 106, 8123-8128). Vanadyl hydrogenphosphate hemihydrate had been precipitated beforehand in n-butanol by boiling under reflux of V₂O₅ and H₃PO₄ (85% p.a., Merck Eurolap GmbH, Darmstadt, Germany).

To prepare the title compound, 69.5 mg of CoO (ultrapure, Merck Eurolap GmbH, Darmstadt, Germany) and 285.7 mg of (VO)₂P₂O₇ were introduced into a corundum crucible. The corundum crucible was closed with gold foil and melted together with 25 mg of PtCl₂ into an evacuated silica glass ampoule. The ampoule was heat-treated isothermally at 1033 K. After five days, the ampoule was taken from the oven and quenched under flowing water. Within the crucible, dark green crystals which had an edge length of about 0.1 mm were present.

The table which follows reports selected characteristic X-ray reflections as obtained by evaluating a Guinier image (FIG. 1).

| 4θ | I | d [Å] |
|---|---|---|
| 28.0927° | 38% | 6.2996 |
| 37.2726° | 42% | 4.7571 |
| 53.7861° | 100% | 3.3124 |
| 56.5972° | 55% | 3.1510 |
| 68.9534° | 22% | 2.5992 |

With reference to a selected crystal with an edge length of 0.1 mm, the space group P2₁/c, Z=2, a6.310(1) Å, b=7.275(1) Å, c=7.441(2) Å, β=90.39(2)° was determined.

### EXAMPLE 2

### Preparation of β-CoV₂O₂(PO₄)₂ by Solid-State Seaction

First, VO₂ was prepared by synproportionation of 181.9 mg of V₂O₅ (p.a., Merck Eurolap GmbH, Darmstadt, Germany) and 149.9 mg of V₂O₃ (from the reduction of V₂O₅ with hydrogen at 1073 K; see G. Brauer, A. Simon in Handbuch der Präparativen Anorganischen Chemie [Handbook of Preparative Inorganic Chemistry], G. Brauer (ed.), Ferd. Enke Verlag, Stuttgart 1981, p. 1419) in closed silica glass ampoules at T=1073 K with addition of 80 mg of iodine as a mineralizer. Cobalt(II) metaphosphate had been obtained beforehand by coevaporation of a nitric acid solution of cobalt(II) nitrate with the stoichiometric amount of phosphoric acid with subsequent calcination of the dry residue at 1023 K under air.

To prepare cobalt(H) vanadium(IV) oxide phosphate according to the equation

Co₂P₄O₁₂+4 VO₂→2 CoV₂O₂(PO₄)₂,

151.3 mg of VO₂ and 199.5 mg of Co₂P₄O₁₂ were introduced into a corundum crucible. This was closed with gold foil and melted together with 25 mg of PtCl₂ into an evacuated silica glass ampoule. The ampoule was heat-treated isothermally at 1073 K.

After five days, the ampoule was taken from the oven and quenched under flowing water. Inside the crucible black crystals were found which, on trituration, gave rise to an olive green powder.

The table which follows reports selected characteristic X-ray reflections as obtained by evaluating a Guinier image (FIG. 2).

| 4θ | I | d [Å] |
|---|---|---|
| 28.1576° | 19% | 6.2851 |
| 37.2467° | 25% | 4.7604 |
| 53.7112° | 100% | 3.3169 |
| 56.6767° | 38% | 3.1467 |
| 68.8403° | 17% | 2.6033 |

### EXAMPLE 3

### Preparation of NiV₂O₂(PO₄)₂ by Solid-State Reaction

The title compound was obtained according to the following equation

NiO+(VO)₂P₂O₇→NiV₂O₂(PO₄)₂.

33 mg of NiO (ultrapure, Merck Eurolap GmbH, Darmstadt, Germany) and 121 mg of (VO)₂P₂O₇ were introduced into a corundum crucible. The crucible was closed with gold foil and melted together with 30 mg of PtCl₂ as a mineralizer into an evacuated silica glass ampoule. The ampoule was heat-treated isothermally at 1033 K. After five days, the ampoule was taken from the oven and quenched under flowing water. Inside the crucible were found black crystals of NiV₂O₂(PO₄)₂. Use of a corundum crucible prevented reaction of the substances with the ampoule wall.

The table which follows reports selected characteristic X-ray reflections as obtained by evaluating a Guinier image (FIG. 3).

| 4θ | I | d [Å] |
|---|---|---|
| 28.129° | 27% | 6.2915 |
| 37.320° | 32% | 4.7512 |
| 53.910° | 100% | 3.3049 |
| 56.728° | 46% | 3.1439 |
| 69.217° | 24% | 2.5896 |

### EXAMPLE 4

### Preparation of CuV₂O₂(PO₄)₂ by Solid-State Reaction

The title compound was obtained according to the following equation

Cu₂P₄O₁₂+4 VO₂→2 CuV₂O₂(PO₄)₂.

VO₂ was prepared as described under Example 2. Copper(II) cyclotetrametaphosphate was obtained by coevaporating a nitric acid solution of copper(II) nitrate with the stoichiometric amount of phosphoric acid and subsequent calcination of the dry residue at 1023 K under air.

160 mg of VO₂ and 213 mg of Cu₂P₄O₁₂ were introduced into a corundum crucible. The crucible was closed with gold foil and melted together with 26 mg of PtCl₂ into an evacuated silica glass ampoule. The ampoule was left at 1013 K in an oven for 5 days. Subsequently, the ampoule taken from the oven was quenched under flowing water. In the corundum crucible was found a black crystalline product, which was CuV₂O₂(PO₄)₂. The trituration of the substance in a mortar gave rise to a black-brown powder.

The table which follows reports selected characteristic X-ray reflections as obtained by evaluating a Guinier image (FIG. 4).

| 4θ | I | d [Å] |
|---|---|---|
| 30.4834° | 30% | 5.8081 |
| 37.1303° | 27% | 4.7752 |
| 38.9490° | 16% | 4.5542 |
| 46.3118° | 14% | 3.8378 |
| 54.3804° | 100% | 3.2769 |
| 56.3670° | 35% | 3.1636 |
| 64.5207° | 10% | 2.7725 |
| 66.3764° | 21% | 2.6971 |

### EXAMPLE 5

### Alternative Preparation of CuV₂O₂(PO₄)₂ by Solid-State Reaction

The title compound was obtained according to the following equation

CuO+(VO)₂P₂O₇→CuV₂O₂(PO₄)₂.

57.0 mg of CuO and 222 mg of (VO)₂P₂O₇ were introduced into a corundum crucible which was closed with gold film. The crucible was melted with 25 mg of PtCl₂ as a mineralizer into an evacuated silica glass ampoule which was then placed into an oven at 1013 K for 6 days. Finally, the ampoule was quenched under flowing water. After it had been opened, black orthorhombic crystals were found in the corundum crucible. Using a selected crystal having an edge length of 0.1 mm, the crystal structure of CuV₂O₂(PO₄)₂ (Pbca, Z=8, a=7.352(1) Å, b=12.652(1) Å, c=14.504(2) Å) was determined with reference to single crystal data.

### EXAMPLE 6

### Preparation of CuV₂O₂(PO₄)₂ by Thermal Degradation of Precursor Compounds

1.03 g of copper(II) acetate monohydrate (Merck Eurolap GmbH, Darmstadt, Germany) were triturated intensively with one another with 1.776 g of vanadyl hydrogenphosphate hemihydrate (for preparation see Example 1) in an Achat mortar. A pellet was then manufactured from the mixture. This pellet was heated in an argon stream first from room temperature to 823 K within 3 hours and left at this temperature for 12 hours. It was then heated to 1013 K within 2 hours and the pellet was left there for 24 hours. Finally, the oven was switched off and the pellet was removed after cooling to about 473 K.

### EXAMPLE 7

### Preparation of α-CoV₂O₂(PO₄)₂ by Thermal Degradation of Precursor Compounds

692.6 mg of cobalt(II) nitrate hexahydrate (Merck Eurolap GmbH, Darmstadt, Germany) and 818.5 mg of vanadyl hydrogenphosphate hemihydrate (for preparation see Example 1) were triturated intensively with one another in an Achat mortar, and a pellet was manufactured. The pellet was heat-treated at 1073 K in an argon stream for 12 hours. After the trituration, the product obtained was an olive green powder, which was microcrystalline α-CoV₂O₂(PO₄)₂.

### EXAMPLE 8

### Preparation of CuV₂O₂(PO₄)₂

The title compound was obtained according to the following equation:

CuCO₃+V₂O₅+H₃PO₃+H₃PO₄→CuV₂O₂(PO₄)₂+CO₂+3 H₂O

6.0 l of water, 545.8 g of V₂O₅ [>99%, 8.25 mol, calculated as V] (GfE Umwelttechnik GmbH, Nuremberg, Germany), 334.4 g of basic CuCO₃ [Cu content 57% by weight, 3 mol, calculated as Cu] (Alfa Aesar Johnson Matthey Management GmbH, Nuremberg, Germany), 345.9 g of H₃PO₄ [85%, 3 mol, calculated as P] (Sigma Aldrich, Seelze, Germany) and 249.7 g of H₃PO₃ [98.5%, 3 mol, calculated as P] (Sigma Aldrich, Seelze, Germany) were introduced into a glass reactor flushed with flowing nitrogen. This mixture was heated to 90° C. with vigorous stirring and stirred at this temperature for 2 hours. Under a nitrogen atmosphere, the suspension thus prepared was dried by means of a spray-dryer (Mobile Minor™ 2000, MM, from Niro A/S, Soborg, Denmark, entrance temperature: 330° C., exit temperature: 107° C.). The resulting solid was calcined in a nitrogen atmosphere in a rotary quartz tube having an internal volume of 1 l at 600° C. for two hours and then at 700° C. for two hours.

The resulting powder had a specific BET surface area of 4.5 m²/g. A powder X-ray diffractogram of the resulting powder was recorded. The following 2θ values were determined from the powder X-ray diffractogram with the accompanying intensities I and interplanar spacings d.

| 2θ | I | d [Å] |
|---|---|---|
| 15.24° | 19% | 5.81 |
| 18.59° | 23% | 4.77 |
| 19.48° | 14% | 4.55 |
| 23.12° | 15% | 3.84 |
| 27.16° | 100% | 3.28 |
| 28.17° | 35% | 3.17 |
| 32.25° | 14% | 2.77 |
| 33.18° | 26% | 2.70 |

### EXAMPLE 9

### Preparation of β-CoV₂O₂(PO₄)₂

The title compound was obtained according to the following equation:

CoCO₃+V₂O₅+H₃PO₃+H₃PO₄>CoV₂O₂(PO₄)₂+CO₂+3 H₂O

6.0 l of water, 545.8 g of V₂O₅ [>99%, 8.25 mol, calculated as V] (GfE Umwelttechnik GmbH, Nuremberg, Germany), 402.9 g of CoCO₃ [Co content 44% by weight, 3 mol, calculated as Co] (Alfa Aesar Johnson Matthey Management GmbH, Nuremberg, Germany), 345.9 g of H₃PO₄ [85%, 3 mol, calculated as P] (Sigma Aldrich, Seelze, Germany) and 251.0 g of H₃PO₃ [98.5%, 3 mol, calculated as P] (Sigma Aldrich, Seelze, Germany) were introduced into a glass reactor flushed with flowing nitrogen. This mixture was heated to 90° C. with vigorous stirring and stirred at this temperature for 2 hours. Under a nitrogen atmosphere, the suspension thus prepared was dried by means of a spray-dryer (Mobile Minor™ 2000, MM, from Niro A/S, Soborg, Denmark, entrance temperature: 330° C., exit temperature: 107° C.). The resulting solid was calcined in a nitrogen atmosphere in a rotary quartz tube having an internal volume of 1 l at 600° C. for two hours and then at 800° C. for two hours.

The resulting powder had a specific BET surface area of 1.6 m²/g. A powder X-ray diffractogram of the resulting powder was recorded. The following 2θ values were determined from the powder X-ray diffractogram with the accompanying intensities I and interplanar spacings d.

| 2θ | I | d [Å] |
|---|---|---|
| 14.13° | 19% | 6.26 |
| 18.69° | 28% | 4.74 |
| 26.87° | 100% | 3.32 |
| 28.43° | 51% | 3.14 |
| 34.44° | 26% | 2.60 |

### EXAMPLE 10

### Preparation of β-NiV₂O₂(PO₄)₂

The title compound was obtained according to the following equation:

NiCO₃+V₂O₅+H₃PO₃+H₃PO₄→NiV₂O₂(PO₄)₂+CO₂+3 H₂O

6.0 l of water, 545.8 g of V₂O₅ [>99%, 8.25 mol, calculated as V] (GfE Umwelttechnik GmbH, Nuremberg, Germany), 177.9 g of NiCO₃ [99%, 3 mol, calculated as Ni] (Alfa Aesar Johnson Matthey Management GmbH, Nuremberg, Germany), 345.9 g of H₃PO₄ [85%, 3 mol, calculated as P] (Sigma Aldrich, Seelze, Germany) and 251.0 g of H₃PO₃ [98.5%, 3 mol, calculated as P] (Sigma Aldrich, Seelze, Germany) were introduced into a glass reactor flushed with flowing nitrogen. This mixture was heated to 90° C. with vigorous stirring and stirred at this temperature for 2 hours, Under a nitrogen atmosphere, the suspension thus prepared was dried by means of a spray-dryer (Mobile Minor™ 2000, MM, from Niro A/S, Soborg, Denmark, entrance temperature: 330° C., exit temperature: 107° C.). The resulting solid was calcined in a nitrogen atmosphere in a rotary quartz tube having an internal volume of 1 l at 600° C. for two hours and then at 750° C. for two hours.

The resulting powder had a specific BET surface area of 1.6 m²/g. A powder X-ray diffractogram of the resulting powder was recorded. The following 2θ values were determined from the powder X-ray diffractogram with the accompanying intensities I and interplanar spacings d.

| 2θ | I | d [Å] |
|---|---|---|
| 14.10° | 18% | 6.27 |
| 18.70° | 29% | 4.74 |
| 26.88° | 100% | 3.31 |
| 28.42° | 56% | 3.14 |
| 34.52° | 26% | 2.60 |

1. A polynary metal oxide phosphate of the general formula I
MₐV₂O_{b}(PO₄)_{c}
in which
M is one or more metals selected from the group consisting of V, Cr, Fe, Co, Ni, Ru, Rh, Pd, Cu, Zn, Cd, Hg, Be, Mg, Ca, Sr and Ba,
a is from 0.5 to 1.5,
b is from 1.5 to 2.5, and
c is from 1.5 to 2.5,
wherein the polynary metal oxide phosphate has having one of the two following crystal structures A or B where
the powder X-ray diffractogram of crystal structure A is characterized by reflections at the interplanar spacings d [Å]=6.28±0.06, 4.75±0.04, 3.31±0.04, 3.14±0.04, 2.60±0.04 and
the powder X-ray diffractogram of crystal structure B is characterized by reflections at interplanar spacings d [Å]=5.81±0.06, 4.77±0.04, 4.55±0.04, 3.84±0.04, 3.28±0.04, 3.17±0.04, 2.77±0.04, 2.70±0.04.

2. The polynary metal oxide phosphate of claim 1, wherein the polynary metal oxide phosphate has the crystal structure A and wherein the reflections have the following relative intensities:

| d [Å] | Rel. intensity [%] |
|---|---|
| 6.28 ± 0.06 | 25 ± 15 |
| 4.75 ± 0.04 | 30 ± 20 |
| 3.31 ± 0.04 | 100 |
| 3.14 ± 0.04 | 45 ± 25 |
| 2.60 ± 0.04 | 25 ± 15 |

3. The polynary metal oxide phosphate of claim 1, wherein the polynary metal oxide phosphate has the crystal structure B and wherein the reflections have the following relative intensities:

| d [Å] | Rel. intensity [%] |
|---|---|
| 5.81 ± 0.06 | 25 ± 15 |
| 4.77 ± 0.04 | 25 ± 15 |
| 4.55 ± 0.04 | 15 ± 10 |
| 3.84 ± 0.04 | 15 ± 10 |
| 3.28 ± 0.04 | 100 |
| 3.17 ± 0.04 | 35 ± 20 |
| 2.77 ± 0.04 | 15 ± 10 |
| 2.70 ± 0.04 | 25 ± 15 |

4. The polynary metal oxide phosphate of claim 1 in which
a is from 0.8 to 1.2,
b is from 1.8 to 2.2, and
c is from 1.8 to 2.2.

5. The polynary metal oxide phosphate of claim 1, wherein M is a metal selected from the group consisting of Co, Ni and Cu.

6. The polynary metal oxide phosphate of claim 5 having any one of the formulas:
CoV₂O₂(PO₄)₂,
NiV₂O₂(PO₄)₂ or
CuV₂O₂(PO₄)₂.

7. A process for preparing the a polynary metal oxide phosphate of claim 1 comprising:
selecting at least two reactants selected from the group consisting of oxygen compounds of vanadium, phosphorus compounds of vanadium and mixed oxygen-phosphorus compounds of vanadium, elemental vanadium, oxygen compounds of the metal M, phosphorus compounds of the metal M and mixed oxygen-phosphorus compounds of the metal M and elemental metal M; and
allowing the selected reactants to react in a sold-state reaction in a closed system.

8. A process for preparing the polynary metal oxide phosphate of claim 1 comprising:
preparing a dry mixture comprising a vanadium source, a source of the metal M and a phosphate source, and
calcining the dry mixture at a temperature of at least 500° C.

9. The process of claim 22, wherein the reduction equivalents are provided by a reducing agent is selected from the group consisting of hypophosphorous acid, phosphorous acid, hydrazine, hydroxylamine, nitrosylamine, elemental vanadium, elemental phosphorus, borane and oxalic acid.

10. The process of claim 8, wherein the dry mixture is prepared by mixing the vanadium source, the source of the metal M, the phosphate group source and a reducing agent in dissolved or suspended form and drying the mixed solution to give the dry mixture.

11. The process of claim 8, wherein the vanadium source is selected from the group consisting of divanadium pentoxide and ammonium vanadate.

12. The process of claim 8, wherein the source of the metal M is selected from the group consisting of nitrates, carboxylates, carbonates, hydrogencarbonates, basic carbonates, oxides, hydroxides and oxide hydroxides of the metal M.

13. The process of claim 8, wherein the phosphate source is formed at least partly by phosphorous acid or hypophosphorous acid.

14. The process of claim 10 13, wherein the drying to give the dry mixture is effected by spray-drying.

15. The process of claim 8, wherein the dry mixture is prepared by mixing vanadyl hydrogenphosphate hemihydrate with a source of the metal M.

16. The process of claim 15, wherein the source of the metal M is selected from the group consisting of nitrates, carboxylates, carbonates, hydrogencarbonates, basic carbonates, oxides, hydroxides and oxide hydroxides of the metal M.

17. A gas phase oxidation catalyst comprising the a polynary metal oxide phosphate of claim 1.

18. The catalyst of claim 17, comprising a first phase and a second phase in the form of three-dimensional delimited regions, the first phase comprising a catalytically active material based on vanadyl pyrophosphate and the second phase comprising the polynary metal oxide phosphate of claim 1.

19. The catalyst of claim 18, wherein finely divided particles of the second phase are dispersed in the first phase.

20. A process for partial gas phase oxidation or ammoxidation comprising contacting a hydrocarbon and molecular oxygen with the catalyst of claim 17.

21. The process of claim 20 for preparing maleic anhydride, wherein the hydrocarbon comprises at least four carbon atoms.

22. The process of claim 8 further comprising providing reduction equivalents in order to convert the vanadium and/or the metal M to the valency state possessed by the vanadium and the metal M in the formula I before the calcining step.

23. The catalyst of claim 18, wherein the first phase and the second phase are distributed relative to one another as in a mixture of finely divided first phase and finely divided second phase.
